(19) United States
(12) Patent Application Publication
(10) Pub. No.: US 2005/0241839 A1
(43) Pub. Date: Nov. 3, 2005

(54) Hand tool with multiple grips

(76) Inventors: David Demar, Tenafly, NJ (US); Thomas VanDyk, Ramsey, NJ (US); Roland Charriez, Mohegan Lake, NY (US); Michael Ballone, New Providence, NJ (US); John Kiely, Morris Plains, NJ (US); Keith Kristiansen, Stratford, CT (US); Melissa Fisher, Columbus, OH (US); Brian J. Conaway, Columbus, OH (US); Mandi Reese, Westerville, OH (US); Terry M. Birchler, New Albany, OH (US); Mark C. Eyman, Columbus, OH (US)

Correspondence Address: Porter, Wright, Morris & Arthur LLP;ATTN: Intellectual Property Department, 28th Floor, 41 South High Street, Columbus, OH, 43215-6194, US

(21) Appl. No.: 11/094,880

(22) Filed: Mar. 30, 2005

Related U.S. Application Data
(60) Provisional application No. 60/567,388, filed on Apr. 30, 2004.

(51) Int. Cl. A61B 17/28

(52) U.S. Cl. 172/378

(57) ABSTRACT

A lawn and garden hand tool includes a tool head, a handle rearwardly extending from the tool head, and a plastic coating covering at least a portion of the handle. The handle has a generally cylindrical shaped rearward grip and a loop extending in a downward direction and connecting forward and rearward ends of the rearward grip. The handle also has a forward grip spaced apart from the rearward grip and located forward of the rearward grip toward the tool head. The forward grip includes a generally bulb shaped knob extending in a upward and forward direction. The tool head, the rearward grip and the forward member are preferably a unitary one-piece cast aluminum component.

### CROSS-REFERENCE TO RELATED APPLICATIONS

This application claims priority benefit from U.S. Provisional Patent Application Number 60/567,388 filed on Apr. 30, 2004, the disclosure of which is expressly incorporated herein in its entirety by reference.

### STATEMENT REGARDING FEDERALLY SPONSORED RESEARCH

Not Applicable

### REFERENCE TO MICROFICHE APPENDIX

Not Applicable

### FIELD OF THE INVENTION

The present invention generally relates to tools and, more particularly, to hand tools for lawn and gardening tasks and the like.

### BACKGROUND OF THE INVENTION

A variety of different hand tools are commonly used for lawn and gardening tasks such as, for example, trowels, rakes, soil knives, cultivators, weeders, hoes, and the like. These hand tools typically have a tool head and a generally straight cylindrically-shaped handle extending rearwardly from the tool head. The tool head is typically metal while the handle is typically wood but is sometimes metal or plastic.

While these prior hand tools may be adequate to perform intended lawn and gardening tasks under some conditions, they can be difficult and uncomfortable to grasp and hold, particularly with two hands, and can require the operator to have a relatively large amount of hand strength. This can particularly be a concern when the hand tools are used by women, elderly, or handicapped users. Additionally, there is a never ending desire to improve certain characteristics of such hand tools such as, for example, quality, reliability, versatility, weather-resistance, high strength, low weight, and low manufacturing costs. Accordingly, there is a need in the art for improved lawn and garden hand tools.

### SUMMARY OF THE INVENTION

The present invention provides a hand tool which overcomes at least some of the above-noted problems of the related art. According to the present invention, a hand tool comprises, in combination, a tool head and a handle rearwardly extending from the tool head. The handle includes a loop handle forming a rearward grip. The handle also includes a forward grip spaced apart from the rearward grip and located forward of the rearward grip toward the tool head.

According to another aspect of the present invention, a hand tool comprises, in combination, a tool head and a handle rearwardly extending from the tool head. The handle has a generally cylindrical shaped rearward grip and a loop extending in a downward direction and connecting forward and rearward ends of the rearward grip. The handle also has a forward grip spaced apart from the rearward grip and located forward of the rearward grip toward the tool head. The forward grip includes a generally bulb shaped knob extending in a upward and forward direction.

According to another aspect of the present invention, a lawn and garden hand tool comprises, in combination, a tool head, a handle rearwardly extending from the tool head, and a plastic coating covering at least a portion of the handle. The handle has a generally cylindrical shaped rearward grip and a loop extending in a downward direction and connecting forward and rearward ends of the rearward grip. The handle also has a forward grip spaced apart from the rearward grip and located forward of the rearward grip toward the tool head. The forward grip includes a generally bulb shaped knob extending in a upward and forward direction. The tool head, the rearward grip and the forward member are preferably a unitary one-piece cast aluminum component.

From the foregoing disclosure and the following more detailed description of various preferred embodiments it will be apparent to those skilled in the art that the present invention provides a significant advance in the technology of hand tools for lawn and garden tasks. Particularly significant in this regard is the potential the invention affords for providing high quality, reliable, versatile, and easy to use hand tools for lawn and gardening tasks with improved operational performance. Additional features and advantages of various preferred embodiments will be better understood in view of the detailed description provided below.

### BRIEF DESCRIPTION OF THE DRAWINGS

These and further features of the present invention will be apparent with reference to the following description and drawings, wherein:

FIG. 1 is a perspective view of a lawn and garden hand tool according to a first preferred embodiment of the present invention wherein a tool head is in the form of a lawn garden trowel;

FIG. 2 is a left-side elevational view of the lawn and garden hand tool of FIG. 1;

FIG. 3 is a top plan view of the lawn and garden hand tool of FIGS. 1 and 2;

FIG. 4 is a front elevational view of the lawn and garden hand tool of FIGS. 1 to 3;

FIG. 5 is a bottom plan view of the lawn and garden hand tool of FIGS. 1 to 4;

FIG. 6 is a cross-sectional view of the lawn and garden hand tool of FIGS. 1 to 5 taken along line 6-6 of FIG. 3;

FIG. 7 is a perspective view of a lawn and garden hand tool according to a second preferred embodiment of the present invention wherein a tool head is in the form of a lawn garden cultivator;

FIG. 8 is a cross-sectional view of the lawn and garden hand tool of FIG. 7 taken along line 8-8 of FIG. 7;

FIG. 9 is a perspective view of a lawn and garden hand tool according to a third preferred embodiment of the present invention wherein a tool head is in the form of a lawn garden weeder;

FIG. 10 is a cross-sectional view of the lawn and garden hand tool of FIG. 9 taken along line 10-10 of FIG. 9;

FIG. 11 is a perspective view of a lawn and garden hand tool according to a fourth preferred embodiment of the present invention wherein a tool head is in the form of a lawn garden transplanter;

FIG. 12 is a cross-sectional view of the lawn and garden hand tool of FIG. 11 taken along line 12-12 of FIG. 11;

FIG. 13 is a perspective view showing one handed operation of the lawn and garden trowel of FIGS. 1 to 6;

FIG. 14 is a perspective view similar to FIG. 13 but showing two handed operation the lawn and garden hand trowel of FIGS. 1 to 6; and

FIG. 15 is a side elevational view similar to FIGS. 13 and 14 but showing one handed operation of the lawn and garden cultivator of FIGS. 7 and 8.

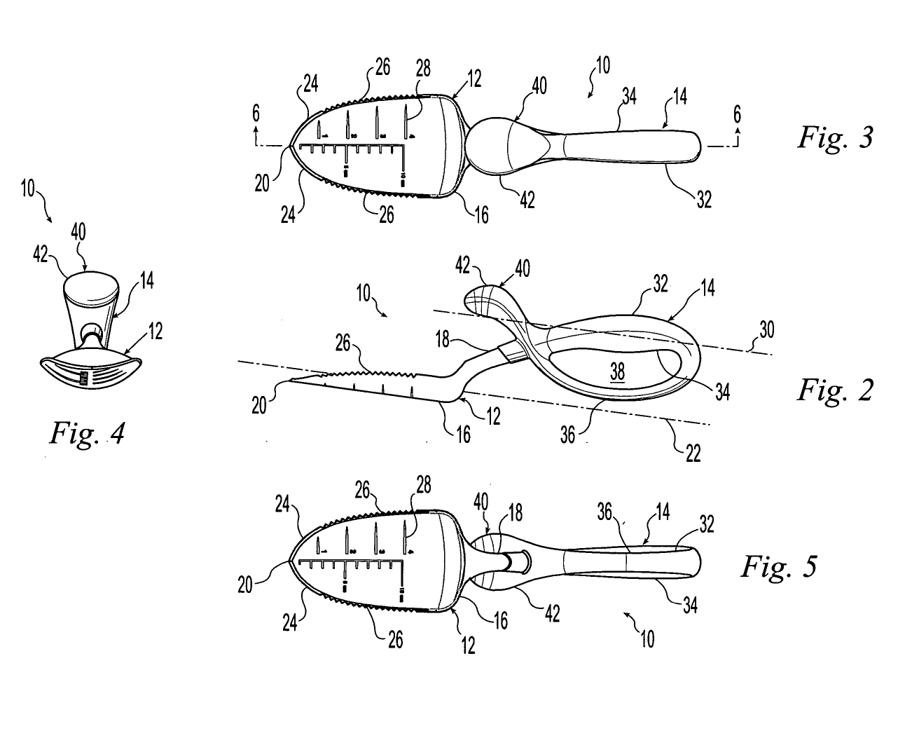

It should be understood that the appended drawings are not necessarily to scale, presenting a somewhat simplified representation of various preferred features illustrative of the basic principles of the invention. The specific design features of the lawn and garden hand tools as disclosed herein, including, for example, specific dimensions, orientations, materials, configurations, and shapes of the various components will be determined in part by the particular intended application and use environment. Certain features of the illustrated embodiments have been enlarged or distorted relative to others to facilitate visualization and clear understanding. In particular, thin features may be thickened, for example, for clarity or illustration. All references to direction and position, unless otherwise indicated, refer to the orientation of the lawn and garden hand tools illustrated in the drawings. In general, up or upward refers to an upward direction within the plane of the paper in the FIG. 2 and down or downward refers to a downward direction within the plane of the paper in the FIG. 2. Also in general, front, fore or forward refers to a direction toward a tool head end of the lawn and garden hand tools which is a leftward direction within the plane of the paper in the FIG. 2 and aft, rear or rearward refers to an handle end of the lawn garden hand tools opposite the tool head end which is a rightward direction within the plane of the paper in FIG. 2.

### DETAILED DESCRIPTION OF CERTAIN PREFERRED EMBODIMENTS

It will be apparent to those skilled in the art, that is, to those who have knowledge or experience in this area of technology, that many uses and design variations are possible for the improved hand tools disclosed herein. The following detailed discussion of various alternative and preferred embodiments will illustrate the general principles of the invention with reference to lawn and garden tools for use in home and garden environments. Other embodiments suitable for other applications will be apparent to those skilled in the art given the benefit of this disclosure.

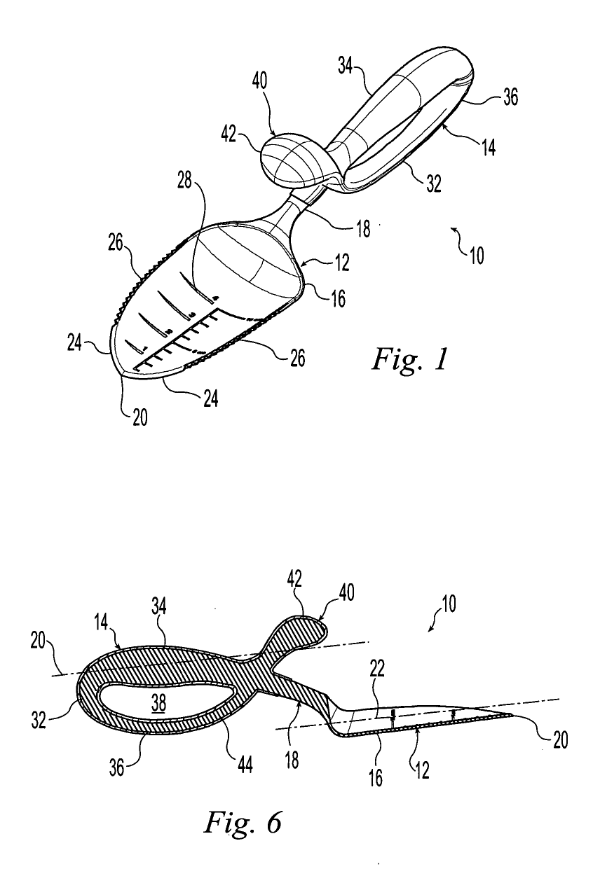

Referring now to the drawings, FIGS. 1 to 6 illustrate a lawn and garden hand tool 10 according to a first preferred embodiment of the present invention. The illustrated hand tool 10 includes a tool head 12 and a handle 14 rearwardly extending from the tool head 12. The illustrated tool head 12 is in the form of a trowel having a main or blade portion 16 and a connecting portion 18. The illustrated blade portion 16 has a forward end which forms a point 20 along a longitudinal axis 22 of the tool head 12. Lateral sides of the illustrated blade portion 16 form cutting edges 24 with teeth or serrations 26 formed thereon. The illustrated blade is curved or arcuate in cross-section so that it is concave in an upward facing direction. The point 20 and the cutting edges 24 are preferably ultra sharp and durable so that desired tasks during use of the hand tool 10 can be performed with less effort and/or hand strength of the user.

The illustrated blade portion 16 includes a scale 28 on the top surface of the blade portion 16 which is configured for indicating the longitudinal distance from the point 20 in order to indicate the depth the blade portion 16 is inserted into the ground. The scale 28 can be formed in the blade portion 16 such as, for example, embossed, debossed, stamped, engraved or otherwise formed on the surface of the blade portion 16 and/or marked on the blade portion 16 such as, for example, painted, printed, stamped, or otherwise marked on the surface of the blade portion 16. The illustrated scale 28 includes measurements in both English and Metric systems but it is noted that the scale 28 can alternatively include any single ore combination of measurement systems.

The connecting portion 18 is sized and shaped for connection of the blade portion 16 to the handle 14. The connecting portion 18 preferably upwardly and rearwardly extends from the blade portion 16 so that a longitudinal axis 30 of the handle 14 is upwardly offset from the longitudinal axis 22 of the blade portion 16. This offset between the axes 22, 30 of the blade portion 16 and the handle 14 provides increased hand clearance for the user during use. For example, the users hand is spaced above the ground when the user moves the hand tool 10 generally parallel to the ground (best shown in FIG. 15). The illustrated blade portion 16 and the illustrated connecting portion 18 are formed as a unitary one-piece component. However, it is noted that the blade portion 16 and the connecting portion 18 can alternatively be formed as separate components rigidly secured together.

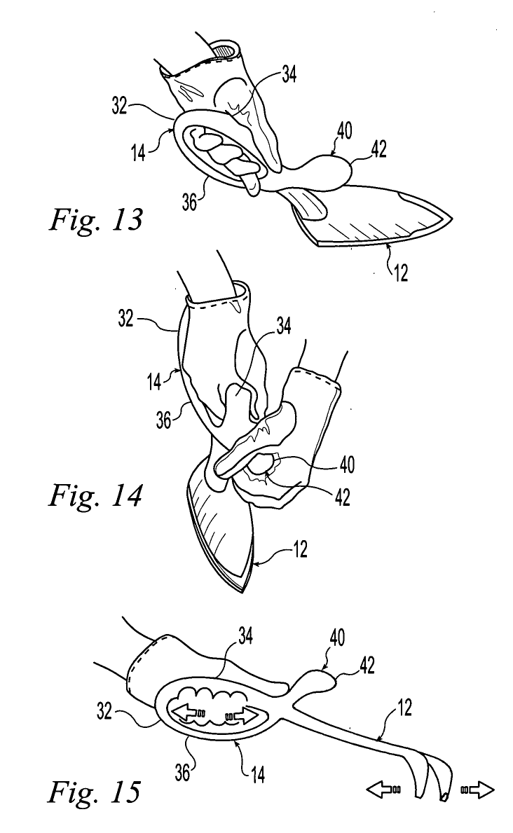

The illustrated handle 14 includes a loop handle 32 forming a first or rearward grip 34 and a loop 36 downwardly extending from the first grip 34. The first grip 34 is elongate and generally cylindrical shaped and extends along the longitudinal axis 30 of the handle 14. The loop 36 connects forward and rearward ends of the first grip 34 and forms a closed, laterally facing opening 38 below and adjacent the first grip 34. The first grip 34 and the opening 38 are sized and shaped so that the first grip 34 can be grasped by a first hand of the user with their fingers passing through the opening 38 so that the hand tool 10 can be used with a single hand of the user (best shown in FIG. 13). It is noted that the opening 38 forms forward and rearward abutments such that the user can move the hand tool 10 in a forward rearward direction with minimal or no grasping of the first grip 34 as the user's hand pushes and pulls the hand tool 10 by engaging the abutments.

The illustrated handle 14 also has a second or forward grip 40 located forward of the first grip 34 space-apart from the first grip 34 and at the forward end of the handle 14. The illustrated second grip 40 includes a knob 42 which extends forwardly and upwardly from the first grip 34 which extends along the longitudinal axis 30. The illustrated second grip knob 42 is generally shaped like a bulb but it is noted that the knob 42 can alternatively have any other suitable shape. The second grip 40 is sized and shaped to be grasped by the second hand of the user to provide a positive comfortable second hand position to deliver added force when needed (best shown in FIG. 14.). It is noted that the first and second grips 34, 40 can each alternatively have any other suitable shape. The first and second grips 34, 40 are each preferably ergonomically shaped or sculpted to provide comfortable hand positions for the user.

The tool head 12 and the handle 14 are each preferably formed of a metal such as, for example, aluminum but can alternatively be formed of any other suitable material such as, for example, steel or plastic. The tool head 12 and the handle 14, including the first and second grips 34, 40, are preferably formed as a unitary one-piece member or component. The illustrated tool head 12 and the illustrated handle 14, including the first and second grips 34, 40, are a unitary one-piece, cast aluminum member or component. It is noted, however, that the tool head 12 and the handle 14 can alternatively be formed as separate components rigidly secured together.

The hand tool 10 preferably includes a covering or coating 44 at least partially covering the handle 14 to provide a comfortable gripping surface for the first and second grips 34, 40. The illustrated coating 44 covers the entire handle 14. The coating 44 preferably comprises plastic and is preferably formed by dipping. The coating 44 is preferably translucent so that the coating 44 is a desired color and the one-piece component forming the tool head 12 and the handle 14 is visible therethrough. However, it is noted that the coating 44 can alternatively be at least partially transparent or opaque

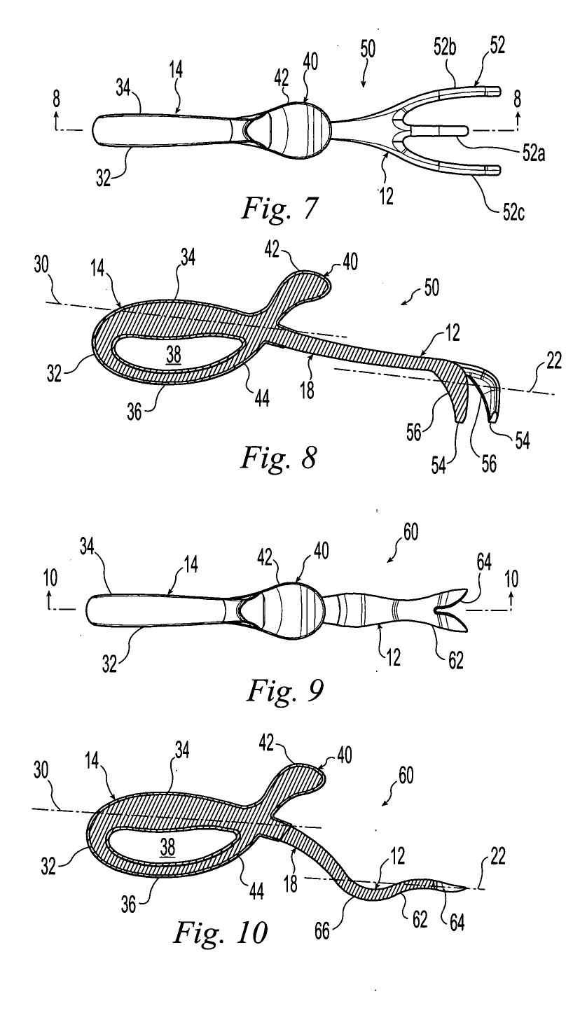

FIGS. 7 and 8 illustrate a lawn and garden hand tool 10 according to a second preferred embodiment of the present invention. The hand tool 50 according to the second preferred embodiment is substantially the same as the hand tool 10 according to the first preferred embodiment except that the tool head 12 is in the form of a cultivator rather than a trowel. Like reference numbers are used to indicate like structure. The hand tool 50 according to the second preferred embodiment illustrates that the tool head 12 can have alternative forms such as, for example, the garden cultivator.

The illustrated cultivator tool head 12 includes a plurality of tines 52 forwardly extending forwardly from the connecting portion 18. The illustrated cultivator tool head 12 includes a central tine 52a and left and right outer tines 52b, 52c spaced laterally from the central tine 52a. The illustrated outer tines 52b, 52c extend further forward than the central tine 52a but each of the tines 52 can alternatively have any other suitable length. Each of the illustrated tines 52 has a downwardly extending point 54 at their forward free ends. Each of the illustrated tines 52 is also provided with a rearward facing cutting edge 56 adjacent the points 54 at the forward free ends of the tines 52. The point 54 and the cutting edges 56 of the tines 52 are preferably ultra sharp and durable so that desired tasks during use of the hand tool 50 can be performed with less effort and/or hand strength of the user.

FIGS. 9 and 10 illustrate a lawn and garden hand tool 60 according to a third preferred embodiment of the present invention. The hand tool 60 according to the third preferred embodiment is substantially the same as the hand tools 10, 50 according to the first and second preferred embodiments except that the tool head 12 is in the form of a weeder rather than a trowel or a cultivator. Like reference numbers are used to indicate like structure. The hand tool 60 according to the third preferred embodiment further illustrates that the tool head 12 can have alternative forms such as, for example, the lawn and garden weeder.

The illustrated weeder tool head 12 includes an elongate blade portion 62 extending forwardly from the connecting portion 18. The illustrated blade portion 62 has a forwardly extending V-shaped notch 64 at its free forward end which is sized and shaped to be placed about the base of a weed. Points and edges of the notch 64 are preferably ultra sharp and durable so that desired tasks during use of the hand tool 60 can be performed with less effort and/or hand strength of the user. The illustrated blade 62 is provided with a bend 66 located between the connecting portion 18 and the free end of the blade portion 62 which is sized and shaped to form a fulcrum for prying weeds placed in the notch 64.

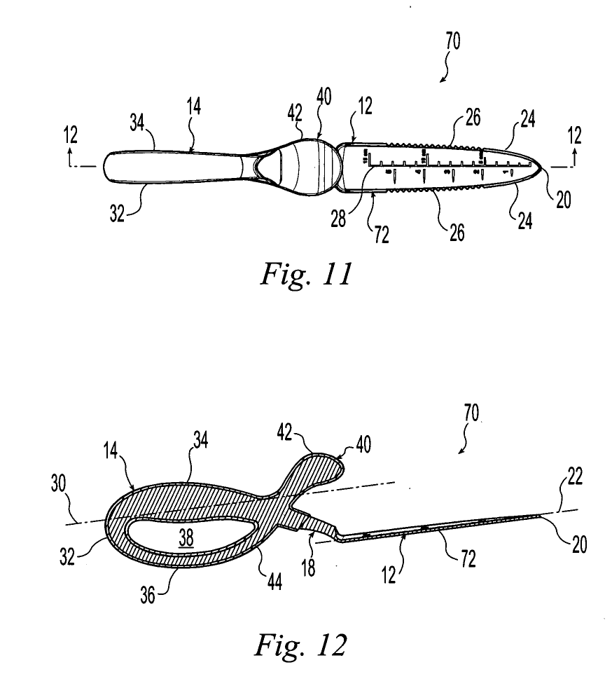

FIGS. 11 and 12 illustrate a lawn and garden hand tool 70 according to a fourth preferred embodiment of the present invention. The hand tool 70 according to the fourth preferred embodiment is substantially the same as the hand tools 10, 50, 60 according to the first to third preferred embodiments except that the tool head 12 is in the form of a transplanter rather than a trowel, a cultivator, or a weeder. Like reference numbers are used to indicate like structure. The hand tool 70 according to the third preferred embodiment even further illustrates that the tool head 12 can have alternative forms such as, for example, the lawn knife.

The illustrated transplanter tool head 12 includes a narrow elongate blade portion 72 extending forwardly from the connecting portion 18. The illustrated transplanter blade portion 72 is substantially the same as the blade portion 16 of the trowel tool head 12 except it is narrower and flatter. The transplanter blade portion 72 is sized and shaped so that the blade portion 72 can be used to precisely cut or dig a plant out of the ground so that it can be transplanted to a new location. The point 20 and the cutting edges 24 of the blade portion 72 are preferably ultra sharp and durable so that desired tasks during use of the hand tool 70 can be performed with less effort and/or hand strength of the user.

It is apparent from the foregoing detailed description that the features of the various embodiments can be used in any combination with the other embodiments. It is also apparent from the foregoing detailed description that the present invention provides improved lawn and garden hand tools which are relatively comfortable to use and require relatively low hand strength. It is further apparent that the improved lawn and garden tools are reliable and versatile and have relatively low manufacturing costs.

From the foregoing disclosure and detailed description of certain preferred embodiments, it will be apparent that various modifications, additions and other alternative embodiments are possible without departing from the true scope and spirit of the present invention. The embodiments discussed were chosen and described to provide the best illustration of the principles of the present invention and its practical application to thereby enable one of ordinary skill in the art to utilize the invention in various embodiments and with various modifications as are suited to the particular use contemplated. All such modifications and variations are within the scope of the present invention as determined by the appended claims when interpreted in accordance with the benefit to which they are fairly, legally, and equitably entitled.

1. A hand tool comprising, in combination:
a tool head;
a handle rearwardly extending from the tool head;
wherein the handle includes a loop handle forming a rearward grip; and
wherein the handle includes a forward grip spaced apart from the rearward grip and located forward of the rearward grip toward the tool head.

2. The hand tool according to claim 1, wherein the forward grip is a knob.

3. The hand tool according to claim 2, wherein the forward grip knob is generally bulb shaped and the rearward grip is generally cylindrical shaped.

4. The hand tool according to claim 2, wherein the forward grip extends in a upward and forward direction.

5. The hand tool according to claim 1, wherein the loop handle has a loop extending in a downward direction and connecting forward and rearward ends of the rearward grip and wherein the loop is sized and shaped to form a closed opening adjacent the rearward grip for passage of a user's hand therethrough when grasping the rearward grip.

6. The hand tool according to claim 1, wherein the tool head is in the form of a trowel.

7. The hand tool according to claim 1, wherein the tool head is in the form of a cultivator.

8. The hand tool according to claim 1, wherein the tool head is in the form of a weeder.

9. The hand tool according to claim 1, wherein the tool head is in the form of a transplanter.

10. The hand tool according to claim 1, wherein the tool head, the rearward grip and the forward member are formed as a unitary one-piece component.

11. The hand tool according to claim 1, wherein the tool head, the rearward grip and the forward grip are cast aluminum.

12. The hand tool according to claim 1, wherein the tool head and handle are formed of metal and the handle is at least partially covered by a plastic coating.

13. A hand tool comprising, in combination:
a tool head;
a handle rearwardly extending from the tool head;
wherein the handle has a generally cylindrical shaped rearward grip;
wherein the handle has a loop extending in a downward direction and connecting forward and rearward ends of the rearward grip;
wherein the handle has a forward grip spaced apart from the rearward grip and located forward of the rearward grip toward the tool head;
wherein the loop is sized and shaped to form a closed opening adjacent the rearward grip for passage of a user's hand therethrough when grasping the rearward grip; and
wherein the forward grip includes a generally bulb shaped knob extending in a upward and forward direction.

14. The hand tool according to claim 13, wherein the tool head is in the form of a trowel.

15. The hand tool according to claim 13, wherein the tool head is in the form of a cultivator.

16. The hand tool according to claim 13, wherein the tool head is in the form of a weeder.

17. The hand tool according to claim 13, wherein the tool head is in the form of a transplanter.

18. The hand tool according to claim 13, wherein the tool head, the rearward grip and the forward member are formed as a unitary one-piece component.

19. The hand tool according to claim 13, wherein the tool head and handle are each formed of metal and the handle is at least partially covered by a plastic coating.

20. A lawn and garden hand tool comprising, in combination:
a tool head;
a handle rearwardly extending from the tool head;
wherein the handle has a generally cylindrical shaped rearward grip;
wherein the handle has a loop extending in a downward direction and connecting forward and rearward ends of the rearward grip;
wherein the loop is sized and shaped to form a closed opening adjacent the rearward grip for passage of a user's hand therethrough when grasping the rearward grip;
wherein the handle has a forward grip spaced apart from the rearward grip and located forward of the rearward grip toward the tool head;
wherein the forward grip includes a generally bulb shaped knob extending in a upward and forward direction;
wherein the tool head, the rearward grip and the forward member are a unitary one-piece cast aluminum component; and
a plastic coating covering at least a portion of the handle.
